(19) United States
(12) Patent Application Publication
(10) Pub. No.: US 2026/0069152 A1
(43) Pub. Date: Mar. 12, 2026

(54) IDENTIFYING SUITABLE CANDIDATES FOR DENERVATION THERAPY

(71) Applicant: Medtronic Ireland Manufacturing Unlimited Company, Dublin (IE)

(72) Inventors: Douglas A. Hettrick, Andover, MN (US); Rakesh Sethi, Vancouver (CA)

(21) Appl. No.: 19/109,478

(22) PCT Filed: Sep. 4, 2023
(86) PCT No.: PCT/EP2023/074116

§ 371 Date: Mar. 6, 2025

Related U.S. Application Data
(60) Provisional application No. 63/374,966, filed on Sep. 8, 2022.

(51) Int. Cl. A61B 5/021; A61B 5/00; G16H 50/30

(52) U.S. Cl. CPC A61B 5/021; A61B 5/4857; A61B 5/681; G16H 50/30

(57) ABSTRACT

An example computing device includes a memory; and one or more processors coupled to the memory, the one or more processors being configured to: determine a baseline circadian pattern of blood pressure for a patient over a first period of time; determine a subsequent circadian pattern of blood pressure for the patient over a second period of time, the second period of time being after the first period of time; determine one or more differences between the subsequent circadian pattern and the baseline circadian pattern; determine, based on the one or more differences between the subsequent circadian pattern and the baseline circadian pattern, whether the patient is a candidate for denervation therapy; and output, responsive to determining whether the patient is a candidate for the denervation therapy, an indication of whether the patient is or is not a candidate for the denervation therapy.

### TECHNICAL FIELD

This application claims the benefit of U.S. Provisional Patent Application Ser. No. 63/374,966, filed Sep. 8, 2022, the entire content of which is incorporated herein by reference.

### TECHNICAL FIELD

This disclosure generally relates to assessing responsiveness to denervation therapy.

### BACKGROUND

Overstimulated or excessively active nerves may result in adverse effects to organs or tissue served by the respective nerves. For example, for some patients, heart, circulatory, or renal disease may be associated with pronounced cardio-renal sympathetic nerve hyperactivity. Stimulation of the renal sympathetic nerves can cause one or more of an increased renin release, increased sodium (Na⁺) reabsorption, or a reduction of renal blood flow. The kidneys may be damaged by direct renal toxicity from the release of sympathetic neurotransmitters (such as norepinephrine) in the kidneys in response to high renal nerve stimulation. Additionally, the increase in release of renin may ultimately increase systemic vasoconstriction, aggravating hypertension.

Percutaneous renal denervation is a procedure that can be used for treating hypertension. During a renal denervation procedure, a clinician delivers stimuli or energy, such as radiofrequency, ultrasound, cooling or other energy, to a treatment site to reduce activity of nerves surrounding a blood vessel. The stimuli or energy delivered to the treatment site may provide various therapeutic effects through alteration of sympathetic nerve activity. Percutaneous denervation of the afferent and efferent renal nerves may result in blood pressure (BP) lowering in some patients with uncontrolled hypertension in both the presence and absence of concomitant anti-hypertensive drug therapy. However, not all patients experience a BP drop immediately following the renal denervation (RDN) procedure, as some patients are “non-responders.”

### SUMMARY

Denervation therapy may provide a therapeutic benefit to certain patients. For example, renal denervation may mitigate symptoms associated with renal sympathetic nerve overactivity. However, not all patients respond well to denervation therapy, such as patients with decreased basal sympathetic nervous system activity. Various aspects of the techniques described in this disclosure may configure a computing device to identify potential non-responders before performing an invasive RDN procedure to deliver denervation therapy that would be ineffective in improving a patient's health. As such, the various aspects of the techniques may reduce performance of potentially ineffective RDN procedures, which in turn improves health care efficiency, while also reducing patient discomfort in undergoing an ineffective (and invasive) RDN procedure to deliver denervation therapy.

The present disclosure describes a non-invasive, accurate and reliable predictor to determine, before performing an RDN procedure to deliver denervation therapy, which potential patients would either respond well to denervation therapy or not respond well to denervation therapy. This predictor may help identify potential non-responders before performing an invasive denervation procedure that would likely end up being ineffective, which may improve health care efficiency and patient care by reducing performance of ineffective procedures while also reducing patient discomfort in undergoing an ineffective procedure.

In one example, this disclosure is directed to a computing device comprising a memory; and one or more processors coupled to the memory, the one or more processors being configured to: determine a baseline circadian pattern of blood pressure for a patient over a first period of time; determine a subsequent circadian pattern of blood pressure for the patient over a second period of time, the second period of time being after the first period of time; determine one or more differences between the subsequent circadian pattern and the baseline circadian pattern; determine, based on the one or more differences between the subsequent circadian pattern and the baseline circadian pattern, whether the patient is a candidate for denervation therapy; and output, responsive to determining whether the patient is a candidate for the denervation therapy, an indication of whether the patient is or is not a candidate for the denervation therapy.

In another example, this disclosure is directed to a system comprising a wearable device configured to sense and collect data indicative of blood pressure of a patient; and a computing device communicatively coupled to the wearable device, wherein the computing device comprises: a memory; and one or more processors coupled to the memory, the one or more processors being configured to: determine a baseline circadian pattern of blood pressure for a patient over a first period of time; determine a subsequent circadian pattern of blood pressure for the patient over a second period of time, the second period of time being after the first period of time; determine one or more differences between the subsequent circadian pattern and the baseline circadian pattern; determine, based on the one or more differences between the subsequent circadian pattern and the baseline circadian pattern, whether the patient is a candidate for denervation therapy; and output, responsive to determining whether the patient is a candidate for the denervation therapy, an indication of whether the patient is or is not a candidate for the denervation therapy.

In another example, this disclosure is directed to a method comprising: determining a baseline circadian pattern of blood pressure for a patient over a first period of time; determining a subsequent circadian pattern of blood pressure for the patient over a second period of time, the second period of time being after the first period of time; determining one or more differences between the subsequent circadian pattern and the baseline circadian pattern; determining, based on the one or more differences between the subsequent circadian pattern and the baseline circadian pattern, whether the patient is a candidate for denervation therapy; and outputting, responsive to determining whether the patient is a candidate for the denervation therapy, an indication of whether the patient is or is not a candidate for the denervation therapy.

Further disclosed herein is a computing device that includes a memory; and one or more processors coupled to the memory, the one or more processors being configured to: determine a baseline circadian pattern of blood pressure for a patient over a first period of time; determine a subsequent circadian pattern of blood pressure for the patient over a second period of time, the second period of time being after the first period of time; determine one or more differences between the subsequent circadian pattern and the baseline circadian pattern; determine, based on the one or more differences between the subsequent circadian pattern and the baseline circadian pattern, whether the patient is a candidate for denervation therapy; and output, responsive to determining whether the patient is a candidate for the denervation therapy, an indication of whether the patient is or is not a candidate for the denervation therapy.

Further details of one or more examples of this disclosure are set forth in the accompanying drawings and in the description below. Other features, objects, and advantages will be apparent from the description and drawings, and from the claims.

The above summary is not intended to describe each illustrated example or every implementation of the present disclosure.

### BRIEF DESCRIPTION OF DRAWINGS

FIG. 1 is a conceptual diagram illustrating an example system for identifying a candidate for denervation therapy, in accordance with some examples of the current disclosure.

FIG. 2 is a block diagram illustrating an example configuration of a data collection device, in accordance with some examples of the current disclosure.

FIGS. 3A-3C are conceptual diagrams of determining and comparing circadian patterns of blood pressure data in accordance with some examples of the current disclosure.

FIG. 3D is a conceptual diagram of a sensed pulsatile waveform in accordance with some examples of the current disclosure.

FIG. 4 is a conceptual diagram illustrating an example neural network configured to predict renal denervation therapy efficacy for a patient diagnosed with hypertension.

FIG. 5 is a flow diagram illustrating an example technique for operating a system to identify suitable patient candidates for denervation therapy.

### DETAILED DESCRIPTION

Denervation therapy, such as renal denervation (RDN) therapy, may be used to render a nerve inert, inactive, or otherwise completely or partially reduced in function, such as by ablation or lesioning of the nerve. Following denervation, there may be a reduction or even prevention of neural signal transmission along the target nerve. Denervating an overactive nerve may provide a therapeutic benefit to a patient. For example, renal denervation may mitigate symptoms associated with renal sympathetic nerve overactivity, such as hypertension. Denervation therapy may include delivering electrical and/or thermal energy to a target nerve, and/or delivering a chemical agent to a target nerve. In the case of renal denervation therapy, the denervation energy or chemical agents can be delivered, for example, via a therapy delivery device (e.g., a catheter) disposed in a blood vessel (e.g., the renal artery) proximate to the renal nerve.

The renal sympathetic nervous system has been identified as a potential major contributor to the complex pathophysiology of hypertension, or elevated systemic blood pressure (BP). Therefore, renal denervation may reduce renal sympathetic nerve overactivity and cause a reduction in systemic BP as a treatment for hypertension. In some patients, renal denervation may reduce systolic BP in a range of approximately 5 millimeters of mercury (mmHg) to 30 mmHg. However, renal denervation may not reduce systolic BP for other patients. RDN therapy may be used as treatment for other ailments associated with changes in sympathetic nervous system activity as well, such as arrhythmias and heart failure.

Efforts to identify candidates who might respond better to the renal denervation therapy have focused in two primary areas. First, identification of patients with potentially higher baseline sympathetic activity as indicated by variables such as increased heart rate, increased plasma renin levels, increased muscle sympathetic nerve activity, increased renal norepinephrine spillover, or the like has been suggested. Examples of increased sympathetic activity include muscles sympathetic nerve activity, renal norepinephrine spillover, response (BP/heart rate) to stimulus (cold, squeeze ball, mental stress), heart rate variability, and biomarkers. However, in some examples, these parameters are either difficult to obtain, have low reproducibility, and/or would be difficult to diligently track over long periods of time.

Second, identification of higher baseline aortic or arterial stiffness due to calcification or other vascular disease that would prevent peripheral arterial vasodilation has also been suggested. Various ways to quantify aortic/arterial stiffness in patients otherwise eligible for renal denervation have been attempted, including imaging based methods of estimating aortic distensibility, invasive determination of pulse wave velocity, noninvasive determination of pulse wave velocity using either two point (Ad/At) processes or morphometry model based single beat estimates, estimates of arterial wave reflection such as an augmentation index, or high arterial pulse pressure (i.e. “isolated systolic hypertension”) identified as high systolic BP with low or normal diastolic BP.

However, none of these ways are accurate and reliable predictors of renal denervation responders. As such, renal denervation is performed in certain situations on patients that do not respond well to renal denervation, resulting in wasted health care resources, patient inconvenience and discomfort, potential side effects, etc.

In some examples, circadian patterns of sympathetic activity may be observed by tracking hemodynamic parameters, such as BP and/or heart rate. Changes in a candidate's circadian pattern of a given hemodynamic index, such as BP, may indicate a change in basal sympathetic nervous system activity. In some examples, a combination of the changes in the circardian pattern and pulse waveform analysis from a baseline reading during a specified period of the circardian pattern may be used to indicate the occurrence of basal sympathetic activity.

The present disclosure describes various aspects of techniques related to determining whether a potential patient for renal denervation (to, for example, reduce hypertension) may be a responder or a non-responder. While the above examples discuss renal denervation responsiveness in reducing hypertension, the techniques described herein may similarly be used to determine renal denervation responsiveness for treatment of other ailments associated with changes in sympathetic nervous system activity, such as arrhythmias and heart failure.

For example, a computing device or system may generate a score indicative of renal denervation responsiveness in reducing hypertension for a patient, where the score is determined based on changes in circadian patterns of a hemodynamic index, such as BP or heart rate. Changes in a candidate's circadian pattern of a given hemodynamic index may indicate a change in basal sympathetic nervous system activity. The changes of a candidate's circadian pattern over a period of time may be compared to a threshold to determine whether a patient may be responsive or non-responsive to renal denervation. One or more parameters of the morphology of the waveform may indicate systemic vascular changes occurring during the circadian pattern such as the time difference between the systolic peak to the dichrotic notch or secondary peak. The pulse waveform analysis may determine changes in arterial compliance over a period of time that may then be compared to a threshold to determine whether a patient may be responsive or non-responsive to renal denervation.

Non-invasively identifying whether a patient may be responsive to denervation therapy, before performing a denervation procedure, to determine whether they will be a candidate for denervation therapy, in accordance with various techniques of this disclosure, may help prevent clinicians from performing invasive denervation procedures on a patient who will not respond to the procedure. This may help reduce unnecessary health care costs by identifying which patients would respond to denervation procedures while also reducing invasive procedures and unnecessary discomfort on non-responsive patients that would be ineffective.

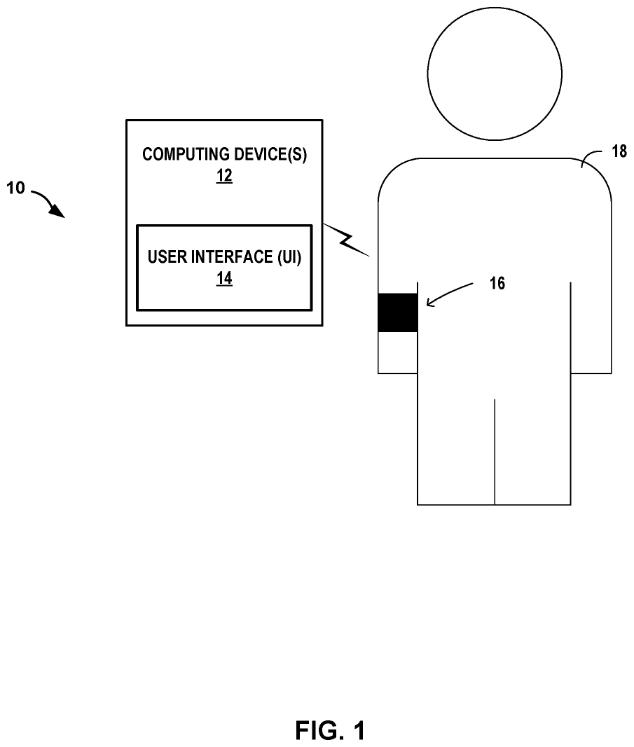

FIG. 1 is a conceptual diagram illustrating an example system 10 for identifying patient candidates for denervation therapy as well as, in some examples, delivering denervation therapy. As shown in FIG. 1, system 10 includes a computing device 12. Computing device 12 may be a computing device used in a home, ambulatory, clinic, or hospital setting. Computing device 14 may include, for example, a clinician programmer, a desktop computer, a laptop computer, a workstation, a server, a mainframe, a cloud computing system, a smartphone, combinations thereof, or the like. Computing device 12 may be configured to receive, via a user interface device 14 (“UI 14”), input from a user, such as a clinician, output information to a user, or both. In some examples, UI 14 may include a display (e.g., a liquid crystal display (LCD) or light emitting diode (LED) display), such as a touch-sensitive display; one or more buttons; one or more keys (e.g., a keyboard); a mouse; one or more dials; one or more switches; a speaker; one or more lights; combinations thereof; or the like.

Computing device 12 may be communicatively coupled to a physiological sensor device 16. In some examples, physiological sensor device 16 may be a wearable medical device, as well as a variety of wearable health or fitness tracking devices. Some examples of physiological signals that may be sensed by such devices may include blood pressure, electrocardiogram (ECG) signals, respiration signals, perfusion signals, activity and/or posture signals, pressure signals, blood oxygen saturation signals, body composition, fluid impedance signals, and blood glucose or other blood constituent signals. In some examples, physiological sensor device 16 may include electrodes configured to contact the skin of the patient, such as patches, watches, rings, necklaces, hearing aids, clothing, car seats, or bed linens. In some examples, physiological sensor device 16 may include electrodes and other sensors to sense physiological signals of patient 18, and may collect and store physiological data and detect episodes based on such signals. In some examples, physiological sensor device 16 may be incorporated into the apparel of patient 18, such as within clothing, shoes, eyeglasses, a watch or wristband, a hat, etc. In some examples, physiological sensor device 16 may be a smartphone, a smartwatch, or other smart apparel. In some examples, physiological sensor device 16 is an accessory or peripheral for a smartphone computing device.

Physiological sensor device 16 may be configured to collect and/or communicate the sensed physiological signals and/or data based on the sensed physiological signals to computing device 12. For examples, physiological sensor device 16 may detect blood pressure values of patient 18 and collect and/or communicate the detected blood pressure values with computing device 12.

With every contraction of the left ventricle of the heart, the left ventricle ejects blood to generate a pressure pulse that travels throughout the arteries of the patient. This pulse is detectable at various locations of a patient, including the wrist of the patient. Sensor(s) 34 may be located in a physiologic sensing device 16, such as an implantable medical device or a wearable device. In some examples, a wearable device may be configured to be placed around a wrist of the patient and the pulse sensor(s) in the wearable device may generate the pulse information representative of the BP pulse. While the BP values generated from the wearable device may have an error range, the error amount may be consistent.

Renal denervation being performed in certain situations on patients that do not respond well to renal denervation, results in wasted health care resources, patient inconvenience and discomfort, potential unnecessary side effects, etc.

In accordance with techniques of the present disclosure, a computing device 12 may determine whether a patient may be responsive or non-responsive to renal denervation based on the circadian pattern of a hemodynamic parameter, such as BP, and the changes in the circadian pattern of the hemodynamic parameter, such as BP, over time.

Computing device 12 may receive hemodynamic parameter(s) of a patient, such as BP, from physiological sensor device 16. In some examples, the hemodynamic parameter may be BP. The physiologic sensing device 16 may be a wearable device. Computing device 12 may determine a baseline circadian pattern of the received hemodynamic parameter(s) for the patient over a first period of time based on the received hemodynamic parameter(s) of the patient over the first period of time. Computing device 12 may determine a subsequent circadian pattern of hemodynamic parameter(s) for the patient over a second period of time based on received hemodynamic parameter(s) of the patient over the second period of time, the second period of time being after the first period of time.

Computing device 12 may determine one or more differences between the baseline circadian pattern of hemodynamic parameter(s) and the subsequent circadian pattern of hemodynamic parameter(s) or pulse waveform changes. Computing device 12 may further determine, based on the one or more differences between the subsequent circadian pattern and the baseline circadian pattern, whether the patient is a candidate for denervation therapy and output, responsive to determining whether the patient is a candidate for the denervation therapy, an indication of whether the patient is or is not a candidate for the denervation therapy.

In some examples, computing device 12 may determine whether the differences between the subsequent circadian pattern and the baseline circadian pattern are greater than a circadian pattern difference threshold. In response to determining the differences between the subsequent circadian pattern and baseline circadian pattern are greater than the circadian pattern difference threshold, computing device 12 may output an indication that the patient is a candidate for a denervation therapy. In response to determining the differences between the subsequent circadian pattern and baseline circadian pattern are less than or equal to the circadian pattern difference threshold, computing device 12 output an indication that the patient is not a candidate for denervation therapy.

In accordance with techniques described above, computing device 12 may provide a non-invasive, accurate and reliable predictor to determine, before performing an RDN procedure to deliver denervation therapy, which potential patients would either respond well to denervation therapy or not respond well to denervation therapy. This predictor may help identify potential non-responders before performing an invasive denervation procedure that would likely end up being ineffective, which may improve health care efficiency and patient care by reducing performance of ineffective procedures while also reducing patient discomfort in undergoing an ineffective procedure.

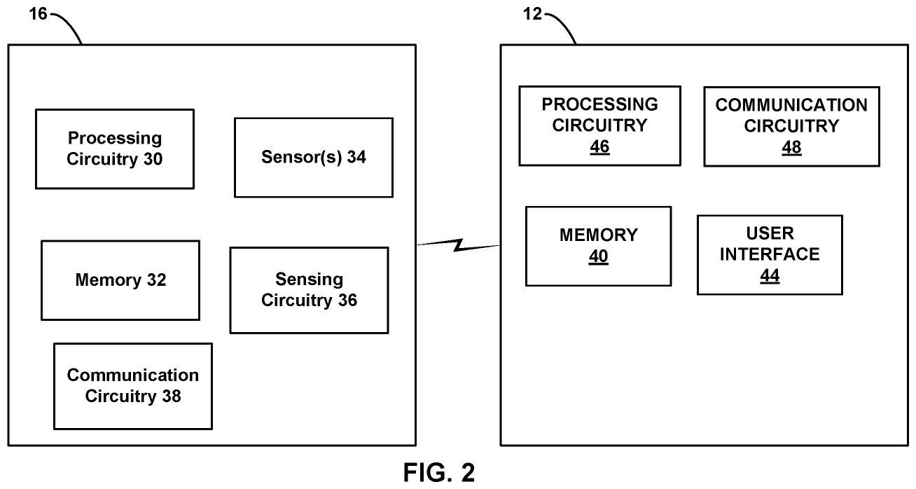

FIG. 2 is a block diagram illustrating an example configuration of physiological sensor device 16 and computing device 12 of FIG. 1. As shown in FIG. 2, physiological sensor device 16 may include processing circuitry 30, memory 32, one or more sensor(s) 34, sensing circuitry 36 coupled to one or more sensor(s) 36, and communication circuitry 38.

One or more sensor(s) 34 of physiological sensor device 16 may sense physiological parameters or signals of patient 18. Sensor(s) 34 may include electrodes, accelerometers (e.g., 3-axis accelerometers), an optical sensor, impedance sensors, temperature sensors, pressure sensors, heart sound sensors (e.g., microphones or accelerometers), and other sensors, and sensing circuitry.

The sensor(s) 34 may detect a pulse and may include any suitable type of sensor, such as spectrophotometric sensors and/or pneumatic pulse sensors, that are configured to generate signals representative of the pulse that may include one or more characteristics of the pulse, such as a pulse rate, a pulse amplitude, a pulse rate variability, a pulse waveform morphology, or a pulse echo. A pulse rate is the rate, or frequency, at which consecutive pulses of blood travel through an artery. The pulse amplitude is the strength or magnitude of the pulse that may be represented as an absolute pressure, relative pressure to atmospheric or baseline pressure, or percentage increase over a baseline amplitude, for example. A pulse rate variability is a representation of how the pulse rate changes over time. A pulse waveform morphology may indicate a shape or feature of the pulse waveform, such as a width of the pulse peak, the slope of the front side and/or back side of the pulse wave, a sharpness of the peak, how many peaks are detected within the pulse wave, one or more notches or interruptions in the pulse wave, or any other feature indicate of the shape of the pulse wave. The pulse echo may be a feature in the pulse wave that is caused by one or more reflected pressure waves within the artery, which may, in some examples, be detected as one or more inflection points in the pulse wave or multiple peaks, humps, or bumps within a single pulse wave. The sensor(s) 34 may transmit the generated pulse information to a pulse monitoring device for conditioning and/or analysis of the pulse information, or the sensor(s) 34 may transmit the pulse information directly to a computing device 12 for patient evaluation.

As also shown in FIG. 2, computing device 12 may include processing circuitry 46, communication circuitry 48, memory 40, and UI 14. Memory 40 may include any volatile or non-volatile media, such as a random access memory (RAM), read only memory (ROM), non-volatile RAM (NVRAM), electrically erasable programmable ROM (EEPROM), flash memory, or the like. Memory 40 may store computer-readable instructions that, when executed by processing circuitry 46, cause computing device 12 to perform various functions described herein. Processing circuitry 46 may include any combination of one or more processors including one or more microprocessors, digital signal processors (DSPs), application specific integrated circuits (ASICs), field programmable gate arrays (FPGAs), or other equivalent integrated or discrete logic circuitry. Accordingly, processing circuitry 46 may include any suitable structure, whether in hardware, software, firmware, or any combination thereof, to perform the functions ascribed herein to processing circuitry 46 and physiological sensor device 16. Computing device 12 may be configured to receive data (e.g., via communication circuitry 368) from physiological sensor device 16.

The sensor(s) 34 may detect a pulse and may include any suitable type of sensor, such as spectrophotometric sensors and/or pneumatic pulse sensors, that are configured to generate signals representative of the pulse that may include one or more characteristics of the pulse, such as a pulse rate, a pulse amplitude, a pulse rate variability, a pulse waveform morphology, or a pulse echo. In some examples, sensor(s) 34 may transmit the generated pulse information to processing circuitry 30 for conditioning and/or analysis of the pulse information, or physiological sensor device 16 may transmit the pulse information, such as via communication circuitry 38, directly to computing device 12.

In some examples, computing device 12 may receive the BP values and/or pulse generated by the sensor(s) 34 in the physiological sensing device 16. The BP values received by computing device 12 may include BP values themselves and/or physiological parameters indicative of the BP values. The received pulse waveform may be the pulse waveform itself and/or physiological parameters indicative of the pulse waveform.

In some examples, the physiological sensing device 16 may be a wearable device, such as a smartwatch. In some examples, computing device 12 may be located in (not shown) the physiological sensing device 16. In some examples, computing device 12 may be separate from the physiological sensing device 16. In some examples, the computing device 12 may generate a BP circadian pattern based on the BP values received from the physiological sensing device 16. In some examples, a circadian pattern of BP may be generated during a first period of time and may be designated as the baseline circadian pattern of BP. A subsequent circadian pattern of BP may be generated over a second period of time, the second period of time being after the first period of time. This use of BP information generated from pulse waves detected from of a patient to determine whether a patient may be responsive or non-responsive to renal denervation is an unconventional use of pulse detection sensors and/or BP detection sensors.

In some examples, the BP circadian pattern may be a circadian pattern of the BP itself and/or a circadian pattern of physiological parameters indicative of the BP values.

The examples herein are directed to human patients. However, the techniques and systems described herein may also be used to screen non-human mammals for renal denervation therapy in other examples.

In accordance with techniques described herein, a computing device may determine a baseline circadian pattern of BP for a patient over a first period of time based on BP signals of the patient over the first period of time, determine a subsequent circadian pattern of BP for the patient over a second period of time based on BP signals of the patient over the second period of time, the second period of time being after the first period of time, and determine a difference between the baseline circadian pattern of BP and the subsequent circadian pattern of BP. In response to the determined difference being less than or equal to a threshold, the computing device may determine basal sympathetic nervous system activity and/or change in basal sympathetic nervous system activity is less than or equal to a threshold, and in response to determining the basal sympathetic nervous system activity and/or change in basal sympathetic nervous system activity is less than or equal to a threshold, determine that the patient is not a candidate for an RDN procedure.

A physiological sensor device 16 may detect blood pressure values and may produce a signal proportional to instantaneous blood pressure variations. In some examples in which the detected blood pressure may include an error or if the physiological sensor device 16 is not properly calibrated, physiological sensor device 16 may be useful for tracking beat to beat variations in the amplitude of systolic blood pressure, including over a 24-hour circadian period.

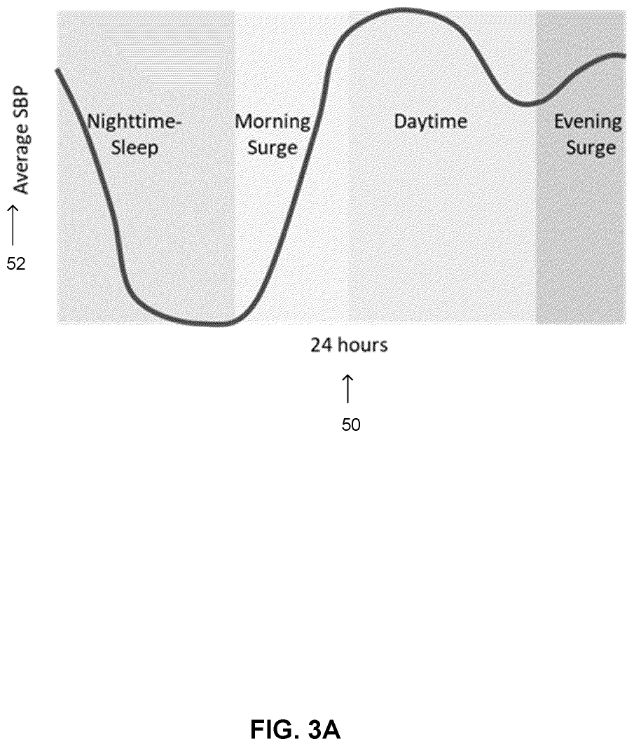

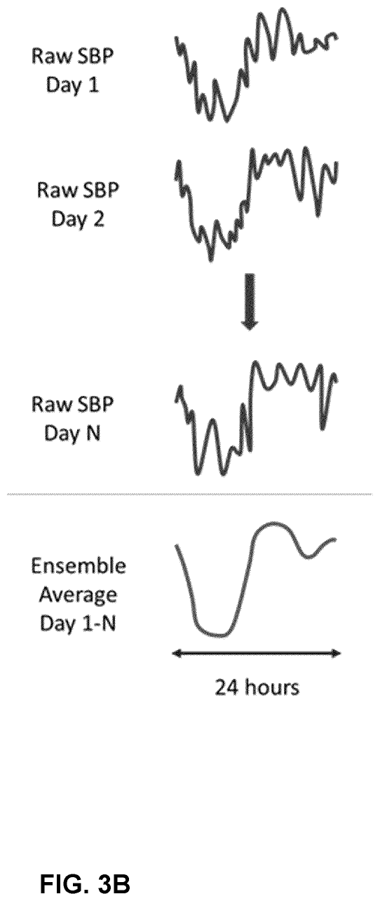

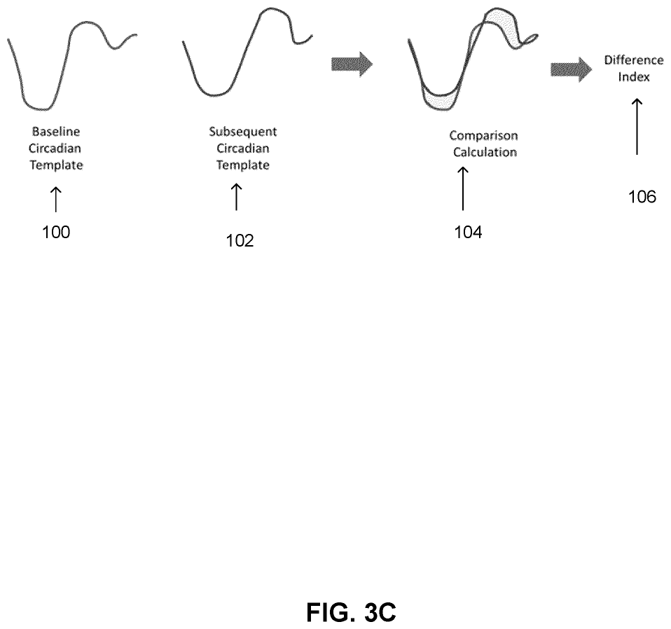

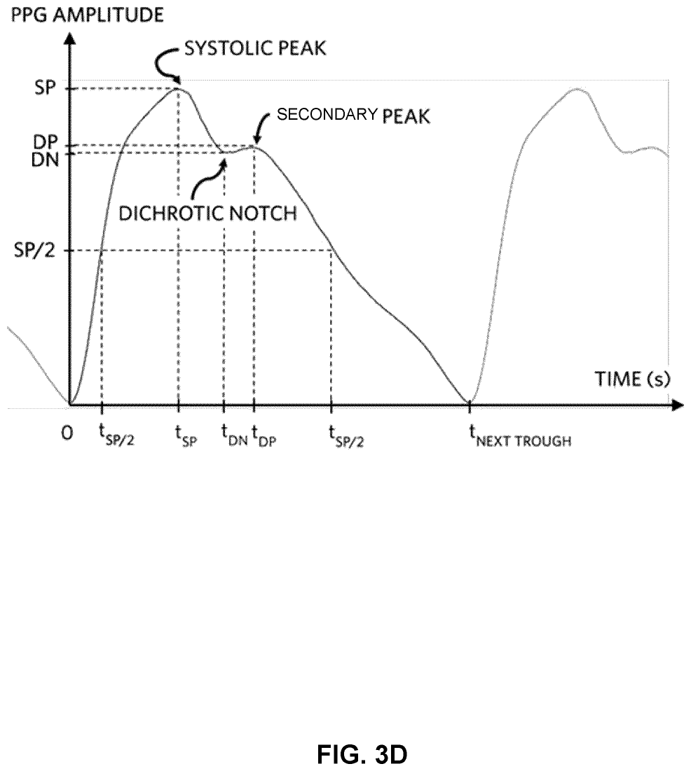

As shown as an example in FIG. 3A, a pattern of blood pressure may vary predictably during a 24-hour circadian cycle with a reduction in the evening at the time of sleep, an abrupt increase in the pre-waking or morning surge, a peak in the late to mid-morning, a relative reduction in the late afternoon, and finally a secondary nighttime surge. In FIG. 3A, the x-axis 50 is a time of a day, and the y-axis 52 is an average systolic blood pressure value at a particular time of the day.

As shown as an example in FIG. 3B, computing device 12 may obtain blood pressure data and/or pulse waveform from day 1 through day N from physiological sensor device 16. In some examples, computing device 12 may receive a plurality of blood pressure measurements over each day, such as every 5 minutes, every 10 minutes, every 30 minutes, every hour, and/or another period of time. In some examples, computing device 12 may not receive consistent blood pressure measurements, but receives a plurality of blood pressure measurements through a 24-hour period of time, such as when physiological sensor device 16 is capable of measuring and sending blood pressure measurements to the computing device. In some examples, as shown in FIG. 3B, the blood pressure measurements received by computing device 12 may be systolic blood pressure measurements. In some examples, the blood pressure measurements received by computing device 12 may be diastolic blood pressure measurements. In some examples, the blood pressure measurements received by computing device 12 may be mean pressure measurements.

Computing device 12 may process the received circadian blood pressure data and/or pulse waveform to determine and generate an ensemble average for the period of time from day 1 to day N. In some examples, this period of time may be one week, two weeks, one month, two months, and/or other periods of time. The days used in the period of time may be adjacent days (e.g., such as Monday through Friday) or may be non-adjacent days (e.g., such as every Monday). In some examples, computing device 12 may generate the ensemble average based on the received blood pressure data and/or pulse waveform to filter out the higher frequency transient variations to reveal an overriding lower frequency circadian pattern.

In some examples, computing device 12 may determine and generate an initial ensemble average of the circadian pattern of blood pressure data and/or pulse waveform over a first period of time as a baseline circadian pattern. In some examples, computing device 12 may generate a baseline circadian pattern in response to receiving an indication from a user, such as a patient and/or a clinician, to generate the baseline circadian pattern.

After generating a baseline circadian pattern, computing device 12 may generate a subsequent circadian pattern over a second period of time. The second period of time happening after the first period of time in which computing device 12 generates the baseline circadian pattern. In some examples, computing device 12 may determine and generate a subsequent circadian pattern similarly to the baseline circadian pattern by receiving blood pressure data and/or pulse waveform over a second period of time and processing the received circadian blood pressure data and/or pulse waveform over the second period of time to determine and generate an ensemble average of the circadian pattern of blood pressure data and/or pulse waveform over the second period of time. In some examples, computing device 12 may identify the ensemble average of the circadian pattern for the second period of time as the subsequent circadian pattern.

The second period of time may be, in some examples, three days, five days, one week, two weeks, one month, two months, and/or other period of time. The days used in the period of time may be adjacent days (e.g., such as Monday through Friday) or may be non-adjacent days (e.g., such as every Monday). In some examples, the second period of time may be updated to be the most recent days in the period of time. For example, if the second period of time is 7 days, when a new day passes and the computing device 12 obtains new blood pressure measurements and/or physiological parameters indicative of the new blood pressure measurements from physiological sensor device 16, the blood pressure measurements from the oldest day in the second period of time is replaced by the blood pressure measurements obtained in the most recent day and the value of the subsequent circadian pattern is updated. Accordingly, in some examples, computing device 12 may update the subsequent circadian pattern value daily while the baseline circadian pattern remains constant. In some examples, computing device may equally weight the blood pressure measurements of each day during the second period of time. In some examples, computing device 12 may weight the blood pressure measurements of each day during the second period of time so the most recent days are more heavily favored.

FIG. 3C shows an example of computing device 12 performing a comparison calculation 104 of a baseline circadian pattern 100 to a subsequent circadian pattern 102 to determine a difference 106 between the baseline circadian pattern to a subsequent circadian pattern. In some examples, the difference between the baseline circadian pattern to a subsequent circadian pattern may be referred to as a difference index 106. The difference between the baseline circadian pattern to the subsequent circadian pattern may indicate a change in basal sympathetic nervous system activity, such as indicating an increase or decrease in basal sympathetic nervous system activity.

FIG. 3D shows an example of a pulsatile waveform sensed by physiological sensor device 16 and components of it. One or more parameters of the morphology of the waveform may indicate arterial compliance, such as the time difference between the systolic peak to the dichrotic notch or secondary peak. In addition, area under the waveform may indicate cardiac output. In some examples, cardiac output may be impacted by stroke volume and/or heart rate. Computing device 12 may perform pulse waveform analysis to determine one or more of changes in the pulse waveform, such as changes in arterial compliance or cardiac output, over a period of time and compare the changes to a respective threshold to determine whether a patient may be responsive or non-responsive to renal denervation.

In some examples, computing device 12 may determine whether differences between the subsequent circadian pattern 102 and baseline circadian pattern 100, such as the difference index 106, are greater than a circadian pattern difference threshold.

In response to determining the differences 106 between the subsequent circadian pattern 102 and baseline circadian pattern 100 are greater than the circadian pattern difference threshold, computing device 12 may output an indication that the patient is a candidate for a denervation therapy. In some examples, in responsive to determining the differences between the subsequent circadian pattern 102 and baseline circadian pattern 100 are greater than the circadian pattern difference threshold, computing device 12 may determine basal sympathetic nervous system activity is greater than a basal sympathetic nervous system activity threshold. In some examples, in response to determining the basal sympathetic nervous system activity is greater than the basal sympathetic nervous system activity threshold, computing device 12 may output the indication that the patient is a candidate for a denervation therapy.

In response to determining the differences 106 between the subsequent circadian pattern 102 and baseline circadian pattern 100 are greater than the circadian pattern difference threshold, computing device 12 may output an indication that the patient is a candidate for a denervation therapy. In some examples, in responsive to determining the differences between the subsequent circadian pattern 102 and baseline circadian pattern 100 are greater than the circadian pattern difference threshold, computing device 12 may determine change in basal sympathetic nervous system activity is greater than a change in basal sympathetic nervous system activity threshold. In response to determining the change in basal sympathetic nervous system activity is greater than the change in basal sympathetic nervous system activity threshold, computing device 12 may output the indication that the patient is a candidate for a denervation therapy.

Computing device 12 may, in response to determining the differences 106 between the subsequent circadian pattern 102 and baseline circadian pattern 100 are less than or equal to the circadian pattern difference threshold, output an indication that the patient is not a candidate for denervation therapy.

In some examples, in response to determining the differences between the subsequent circadian pattern and baseline circadian pattern are less than or equal to the circadian pattern difference threshold, computing device 12 may determine the basal sympathetic nervous system activity is less than or equal to the basal sympathetic nervous system activity threshold. In some examples, in response to determining the basal sympathetic nervous system activity is less than or equal to the basal sympathetic nervous system activity threshold, computing device 12 may output the indication that the patient is a not candidate for denervation therapy.

In response to determining the differences 106 between the subsequent circadian pattern 102 and baseline circadian pattern 100 are less than or equal to the circadian pattern difference threshold, computing device 12 may determine the change in basal sympathetic nervous system activity is less than or equal to the change in basal sympathetic nervous system activity threshold. In response to determining the change in basal sympathetic nervous system activity is less than or equal to the change in basal sympathetic nervous system activity threshold, computing device 12 may output the indication that the patient is a not candidate for denervation therapy.

In some examples, non-invasively identifying whether a patient may be responsive to denervation therapy, before performing a denervation procedure, to determine whether they will be a candidate for denervation therapy may help prevent clinicians from performing invasive denervation procedures on a patient who will not respond to the procedure. This may help reduce unnecessary health care costs by identifying which patients would respond to denervation procedures while also reducing invasive procedures on non-responsive patients that would be ineffective.

Computing device 12 may be configured to execute an AI engine that operates according to one or more models, such as machine learning models. Machine learning models may include any number of different types of machine learning models, such as neural networks, deep neural networks, dense neural networks, and the like. Although described with respect to machine learning models, the techniques described in this disclosure are also applicable to other types of AI models, including rule-based models, finite state machines, and the like.

Machine learning may generally enable a computing device to analyze input data and identify an action to be performed responsive to the input data. Each machine learning model may be trained using training data that reflects likely input data. The training data may be labeled or unlabeled (meaning that the correct action to be taken based on a sample of training data is explicitly stated or not explicitly stated, respectively).

The training of the machine learning model may be guided (in that a designer, such as a computer programmer, may direct the training to guide the machine learning model to identify the correct action in view of the input data) or unguided (in that the machine learning model is not guided by a designer to identify the correct action in view of the input data). In some instances, the machine learning model is trained through a combination of labeled and unlabeled training data, a combination of guided and unguided training, or possibly combinations thereof. Examples of machine learning include nearest neighbor, naïve Bayes, decision trees, linear regression, support vector machines, neural networks, k-Means clustering, Q-learning, temporal difference, deep adversarial networks, evolutionary algorithms or other supervised, unsupervised, semi-supervised, or reinforcement learning algorithms to train one or more models.

Computing device 12 may utilize machine learning, such as a deep learning algorithm or model (e.g., a neural network or deep belief network), to generate a score indicative of whether a patient 18 may be responsive or non-responsive to renal denervation to determine whether a patient 18 may be responsive or non-responsive to renal denervation. The generated score may be output as an indication of whether the patient is or is not a candidate for the denervation therapy. Computing device 12 may train a deep learning model to represent a relationship of changes in circadian patterns of BP and other patient metrics of patients to the responsiveness of a patient to renal denervation therapy. For example, computing device 12 may train the deep learning model using changes in circadian patterns of BP, patient metrics, and renal denervation responsiveness from other patients. In some examples, computing device 12 may train the deep leaning model by adjusting the weights of a hidden layer of a neural network model to balance the contribution of each input (e.g., characteristics of the changes in circadian patterns of BP, pulse waveform, and/or the values of each patient metric) according to how responsive a patient was renal denervation therapy.

Once the deep learning model is trained, computing device 12 may obtain and apply data, such as the circadian patterns of BP for a patient and a plurality of values representative of respective patient metrics for the patient, to the trained deep learning model. Example patient metrics may include an age of the patient, a gender of the patient, an ethnic background of the patient, a weight of the patient, a height of the patient, a diet of the patient, an activity level of the patient, or a stress level of the patient. The circadian patterns of BP may include a baseline circadian pattern of BP and one or more subsequent circadian patterns of BP. The output of the deep learning model may include the score that indicates whether or not the patient would be a responsive to renal denervation therapy. For example, the score may be a probability that the patient would achieve a target reduction in hypertension in response to receiving renal denervation therapy. In some examples, the score may be indicative of the magnitude of the reduction in systemic blood pressure that the patient may realize after renal denervation therapy. Computing device 12 may display the score to a clinician to aid in determining whether or not the patient should receive renal denervation therapy.

In some examples, computing device 12 may send the received BP measurements, such as the baseline circadian pattern of BP and one or more subsequent circadian patterns of BP, to a separate computing system to utilize the separate computing system to perform the machine learning, such as a deep learning algorithm or model, on the received BP measurements, such as the baseline circadian pattern of BP and one or more subsequent circadian patterns of BP.

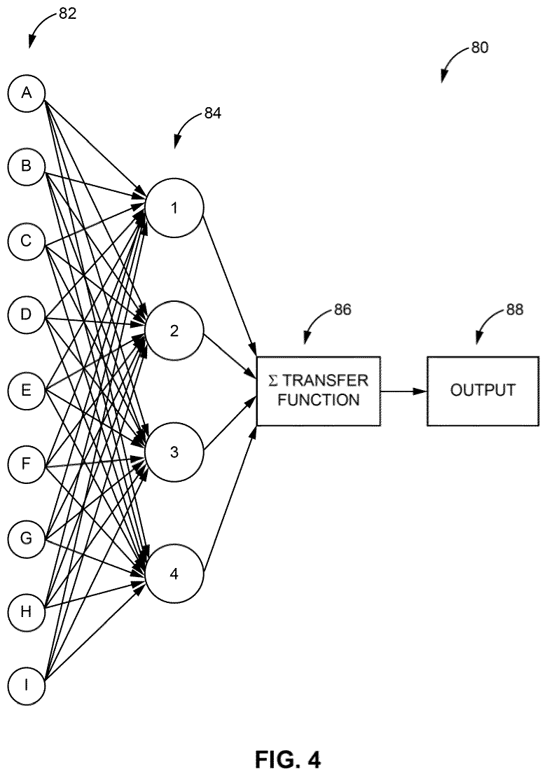

FIG. 4 is a conceptual diagram illustrating an example neural network 80 configured to predict renal denervation therapy efficacy for a patient diagnosed with hypertension. Neural network 80 is an example of a deep learning model, or deep learning algorithm, trained to generate a score indicative of renal denervation therapy efficacy. As discussed above, other types of machine learning and deep learning models or algorithms may be utilized in other examples. Computing device 12 may train, store, and/or utilize neural network 80, but other devices may apply inputs associated with a particular patient to neural network 80 in other examples. Neural network 80 is an example of neural network 58 (FIG. 3), which may be stored by computing device 12.

As shown in the example of FIG. 4, neural network 80 comprises three layers. These three layers include input layer 82, hidden layer 84, and output layer 86. Output layer 88 comprises the output from the transfer function of output layer 86. Input layer 82 represents each of the input values A through I provided to neural network 80. The input values may be values for patient metrics such as an age of the patient, a gender of the patient, an ethnic background of the patient, a weight of the patient, a height of the patient, a diet of the patient, an activity level of the patient, and/or a stress level of the patient. The input values may be numerical or categorical as appropriate for each patient metric. In some examples, values for all of these patient metrics may be incorporated into neural network 80.

In addition, some input values of input layer 82 may include one or more characteristics of hemodynamic information, such as blood pressure information, from the physiological sensing device 16 and/or computing device 12. For example, the characteristics may include BP circadian patterns based on the BP values received from the physiological sensing device 16, such as a baseline circadian pattern, or subsequent circadian pattern. These characteristics may be input into input layer 82. In some examples, the characteristics, such as pulse waveform morphology may be converted to a numerical value or some other input representative of the type of waveform identified from the pulse wave of the blood in the patient.

Each of the input values for each node in the input layer 82 is provided to each node of hidden layer 84. In the example of FIG. 4, hidden layer 84 include four nodes, but fewer or greater number of nodes may be used in other examples. Each input from input layer 82 is multiplied by a weight and then summed at each node of hidden layer 84. During training of neural network 80, the weights for each input are adjusted to establish the relationship between the BP circadian patterns based on the BP values to renal denervation efficacy. In some examples, two or more hidden layers may be incorporated into neural network 80, where each layer includes the same or different number of nodes.

The result of each node within hidden layer 84 is applied to the transfer function of output layer 86. The transfer function may be liner or non-linear, depending on the number of layers within neural network 80. Example non-linear transfer functions may be a sigmoid function or a rectifier function. The output 88 of the transfer function may be the score that is generated by computing device 12 in response to applying the BP circadian patterns based on the BP values for the patient to neural network 80. A deep learning model, such as neural network 80, may enable a computing system such as computing device 12 to screen patients for renal denervation therapy using a variety of values representing the condition of a particular patient.

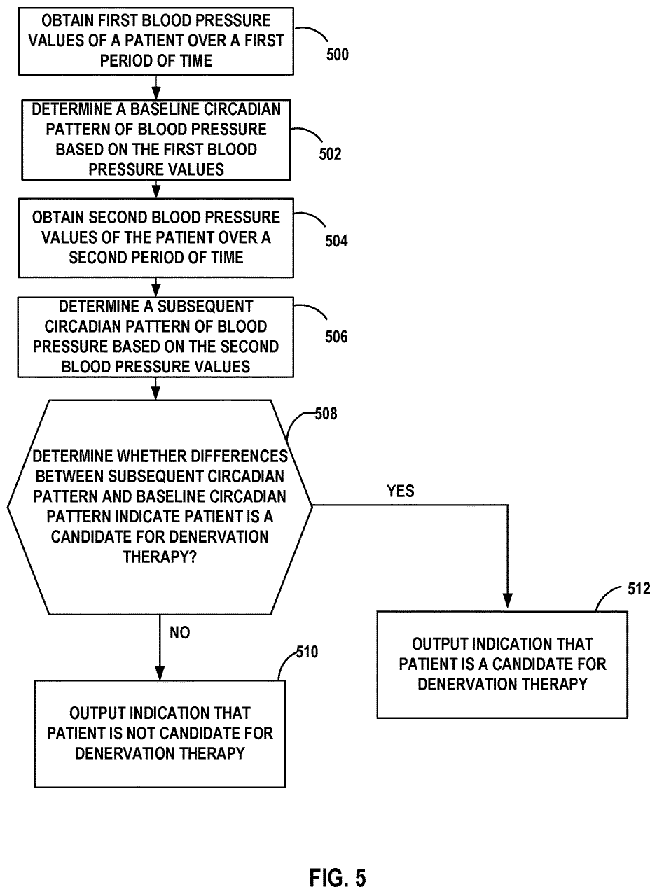

FIG. 5 is a flow diagram illustrating an example technique for operating system 10. As indicated by FIG. 5 computing device 12 may obtain first blood pressure values and/or physiological parameters indicative of the first blood pressure values of a patient over a first period of time (500). Computing device 12 may determine a baseline circadian pattern of blood pressure based on the first blood pressure values (502). Computing device 12 may obtain second blood pressure values and/or physiological parameters indicative of the second blood pressure values of the patient over a second period of time, the second period of time being after the first period of time (504). Computing device 12 may determine a subsequent circadian pattern of blood pressure based on the second blood pressure values (506). Computing device 12 may determine whether differences between the subsequent circadian pattern and baseline circadian pattern indicate the patient is a candidate for denervation therapy (508). In response to computing device 12 determining the differences between the subsequent circadian pattern and baseline circadian pattern indicate the patient is not a candidate for denervation therapy, computing device 12 may output an indication that the patient is not a candidate for a denervation therapy (510). In response to computing device 12 determining the differences between the subsequent circadian pattern and baseline circadian pattern indicate the patient is a candidate for denervation therapy, computing device 12 may output an indication that the patient is a candidate for denervation therapy (512).

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various aspects of the techniques may enable the following examples.

Example 1: A computing device includes a memory; and one or more processors coupled to the memory, the one or more processors being configured to: determine a baseline circadian pattern of blood pressure for a patient over a first period of time; determine a subsequent circadian pattern of blood pressure for the patient over a second period of time, the second period of time being after the first period of time; determine one or more differences between the subsequent circadian pattern and the baseline circadian pattern; determine, based on the one or more differences between the subsequent circadian pattern and the baseline circadian pattern, whether the patient is a candidate for denervation therapy; and output, responsive to determining whether the patient is a candidate for the denervation therapy, an indication of whether the patient is or is not a candidate for the denervation therapy.

Example 2: The computing device of example 1, wherein the one or more processors are further configured to: obtain one or more of the first blood pressure values or physiological parameters indicative of the first blood pressure values of the patient over the first period of time from a sensing device; and obtain one or more of the second blood pressure values or physiological parameters indicative of the second blood pressure values of the patient over the second period of time from the sensing device.

Example 3: The computing device of any of examples 1 through 2, wherein the one or more processors are further configured to: determine whether the differences between the subsequent circadian pattern and the baseline circadian pattern are greater than a circadian pattern difference threshold; responsive to determining the differences between the subsequent circadian pattern and baseline circadian pattern are greater than the circadian pattern difference threshold, output an indication that the patient is a candidate for a denervation therapy; and responsive to determining the differences between the subsequent circadian pattern and baseline circadian pattern are less than or equal to the circadian pattern difference threshold, output an indication that the patient is not a candidate for denervation therapy.

Example 4: The computing device of any of examples 1 through 3, wherein the one or more processors are further configured to: responsive to determining the differences between the subsequent circadian pattern and baseline circadian pattern are greater than the circadian pattern difference threshold, determine one or more of basal sympathetic nervous system activity is greater than a basal sympathetic nervous system activity threshold or change in basal sympathetic nervous system activity is greater than a change in basal sympathetic nervous system activity threshold; responsive to determining the differences between the subsequent circadian pattern and baseline circadian pattern are less than or equal to the circadian pattern difference threshold, determine one or more of the basal sympathetic nervous system activity is less than or equal to the basal sympathetic nervous system activity threshold or the change in basal sympathetic nervous system activity is less than or equal to the change in basal sympathetic nervous system activity threshold; responsive to determining one or more of the basal sympathetic nervous system activity is greater than the basal sympathetic nervous system activity threshold or the change in basal sympathetic nervous system activity is greater than the change in basal sympathetic nervous system activity threshold, output the indication that the patient is a candidate for a denervation therapy; and responsive to determining one or more of the basal sympathetic nervous system activity is less than or equal to the basal sympathetic nervous system activity threshold or the change in basal sympathetic nervous system activity is less than or equal to the change in basal sympathetic nervous system activity threshold, output the indication that the patient is a not candidate for denervation therapy.

Example 5: The computing device of any of examples 1 through 4, wherein the first period of time is one week, two weeks, or one month.

Example 6: The computing device of any of examples 1 through 5, wherein the second period of time is three days, five days, one week, or two weeks.

Example 7: The computing device of any of examples 1 through 6, wherein features of the baseline circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability, and features of the subsequent circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability.

Example 8: The computing device of example 7, wherein the one or more processors are further configured to compare one or more of the features of the baseline circadian pattern to a respective one or more of the features of the subsequent circadian pattern to determine the one or more differences between the subsequent circadian pattern and the baseline circadian pattern.

Example 9: The computing device of any of examples 1 through 8, wherein the first blood pressure values or physiological parameters indicative of the first blood pressure values and the second blood pressure values or physiological parameters indicative of the second blood pressure values are obtained from a wearable device.

Example 10: The computing device of any of examples 1 through 9, wherein the first blood pressure values and the second blood pressure values are systolic blood pressure values.

Example 11: A system includes a wearable device configured to sense and collect data indicative of blood pressure of a patient; and a computing device communicatively coupled to the wearable device, wherein the computing device comprises: a memory; and one or more processors coupled to the memory, the one or more processors being configured to: determine a baseline circadian pattern of blood pressure for a patient over a first period of time; determine a subsequent circadian pattern of blood pressure for the patient over a second period of time, the second period of time being after the first period of time; determine one or more differences between the subsequent circadian pattern and the baseline circadian pattern; determine, based on the one or more differences between the subsequent circadian pattern and the baseline circadian pattern, whether the patient is a candidate for denervation therapy; and output, responsive to determining whether the patient is a candidate for the denervation therapy, an indication of whether the patient is or is not a candidate for the denervation therapy.

Example 12: The system of example 11, wherein the one or more processors are further configured to: obtain one or more of the first blood pressure values or physiological parameters indicative of the first blood pressure values of the patient over the first period of time from a sensing device; and obtain one or more of the second blood pressure values or physiological parameters indicative of the second blood pressure values of the patient over the second period of time from the sensing device.

Example 13: The system of any of examples 11 through 12, wherein the one or more processors are further configured to: determine whether the differences between the subsequent circadian pattern and the baseline circadian pattern are greater than a circadian pattern difference threshold; responsive to determining the differences between the subsequent circadian pattern and baseline circadian pattern are greater than the circadian pattern difference threshold, output an indication that the patient is a candidate for a denervation therapy; and responsive to determining the differences between the subsequent circadian pattern and baseline circadian pattern are less than or equal to the circadian pattern difference threshold, output an indication that the patient is not a candidate for denervation therapy.

Example 14: The system of any of examples 11 through 13, wherein the one or more processors are further configured to: responsive to determining the differences between the subsequent circadian pattern and baseline circadian pattern are greater than the circadian pattern difference threshold, determine one or more of basal sympathetic nervous system activity is greater than a basal sympathetic nervous system activity threshold or change in basal sympathetic nervous system activity is greater than a change in basal sympathetic nervous system activity threshold; responsive to determining the differences between the subsequent circadian pattern and baseline circadian pattern are less than or equal to the circadian pattern difference threshold, determine one or more of the basal sympathetic nervous system activity is less than or equal to the basal sympathetic nervous system activity threshold or the change in basal sympathetic nervous system activity is less than or equal to the change in basal sympathetic nervous system activity threshold; responsive to determining one or more of the basal sympathetic nervous system activity is greater than the basal sympathetic nervous system activity threshold or the change in basal sympathetic nervous system activity is greater than the change in basal sympathetic nervous system activity threshold, output the indication that the patient is a candidate for a denervation therapy; and responsive to determining one or more of the basal sympathetic nervous system activity is less than or equal to the basal sympathetic nervous system activity threshold or the change in basal sympathetic nervous system activity is less than or equal to the change in basal sympathetic nervous system activity threshold, output the indication that the patient is a not candidate for denervation therapy.

Example 15: The system of any of examples 11 through 13, wherein the first period of time is one week, two weeks, or one month.

Example 16: The system of any of examples 11 through 15, wherein the second period of time is three days, five days, one week, or two weeks.

Example 17: The system of any of examples 11 through 16, wherein features of the baseline circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability, and features of the subsequent circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability.

Example 18: The system of example 17, wherein the one or more processors are further configured to compare one or more of the features of the baseline circadian pattern to a respective one or more of the features of the subsequent circadian pattern to determine the one or more differences between the subsequent circadian pattern and the baseline circadian pattern.

Example 19: The system of any of examples 11 through 18, wherein the first blood pressure values or physiological parameters indicative of the first blood pressure values and the second blood pressure values or physiological parameters indicative of the second blood pressure values are obtained from a wearable device.

Example 20: The system of any of examples 11 through 19, wherein the first blood pressure values and the second blood pressure values are systolic blood pressure values.

Example 21: A method includes determining a baseline circadian pattern of blood pressure for a patient over a first period of time; determining a subsequent circadian pattern of blood pressure for the patient over a second period of time, the second period of time being after the first period of time; determining one or more differences between the subsequent circadian pattern and the baseline circadian pattern; determining, based on the one or more differences between the subsequent circadian pattern and the baseline circadian pattern, whether the patient is a candidate for denervation therapy; and outputting, responsive to determining whether the patient is a candidate for the denervation therapy, an indication of whether the patient is or is not a candidate for the denervation therapy.

Example 22: The method of example 21, wherein the method further comprises: obtaining one or more of the first blood pressure values or physiological parameters indicative of the first blood pressure values of the patient over the first period of time from a sensing device; and obtaining one or more of the second blood pressure values or physiological parameters indicative of the second blood pressure values of the patient over the second period of time from the sensing device.

Example 23: The method of any of examples 21 through 22, wherein the method further comprises: determining whether the differences between the subsequent circadian pattern and the baseline circadian pattern are greater than a circadian pattern difference threshold; responsive to determining the differences between the subsequent circadian pattern and baseline circadian pattern are greater than the circadian pattern difference threshold, outputting an indication that the patient is a candidate for a denervation therapy; and responsive to determining the differences between the subsequent circadian pattern and baseline circadian pattern are less than or equal to the circadian pattern difference threshold, outputting an indication that the patient is not a candidate for denervation therapy.

Example 24: The method of examples 21 through 23, wherein the method further comprises: responsive to determining the differences between the subsequent circadian pattern and baseline circadian pattern are greater than the circadian pattern difference threshold, determining one or more of basal sympathetic nervous system activity is greater than a basal sympathetic nervous system activity threshold or change in basal sympathetic nervous system activity is greater than a change in basal sympathetic nervous system activity threshold; responsive to determining the differences between the subsequent circadian pattern and baseline circadian pattern are less than or equal to the circadian pattern difference threshold, determining one or more of the basal sympathetic nervous system activity is less than or equal to the basal sympathetic nervous system activity threshold or the change in basal sympathetic nervous system activity is less than or equal to the change in basal sympathetic nervous system activity threshold; responsive to determining one or more of the basal sympathetic nervous system activity is greater than the basal sympathetic nervous system activity threshold or the change in basal sympathetic nervous system activity is greater than the change in basal sympathetic nervous system activity threshold, outputting the indication that the patient is a candidate for a denervation therapy; and responsive to determining one or more of the basal sympathetic nervous system activity is less than or equal to the basal sympathetic nervous system activity threshold or the change in basal sympathetic nervous system activity is less than or equal to the change in basal sympathetic nervous system activity threshold, outputting the indication that the patient is a not candidate for denervation therapy.

Example 25: The method of any of examples 21 through 24, wherein the first period of time is one week, two weeks, or one month.

Example 26: The method of any of examples 21 through 25, wherein the second period of time is three days, five days, one week, or two weeks.

Example 27: The method of any of examples 21 through 26, wherein features of the baseline circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability, and features of the subsequent circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability.

Example 28: The method of example 27, wherein the method further comprises comparing one or more of the features of the baseline circadian pattern to a respective one or more of the features of the subsequent circadian pattern to determine the one or more differences between the subsequent circadian pattern and the baseline circadian pattern.

Example 29: The method of any of examples 21 through 28, wherein the first blood pressure values or physiological parameters indicative of the first blood pressure values and the second blood pressure values or physiological parameters indicative of the second blood pressure values are obtained from a wearable device.

Example 30: The method of any of examples 21 through 29, wherein the first blood pressure values and the second blood pressure values are systolic blood pressure values.

Various examples have been described. These and other examples are within the scope of the following claims.

Further disclosed herein is the subject-matter of the following clauses:
1. A computing device comprising:
   a memory; and
   one or more processors coupled to the memory, the one or more processors being configured to:
      determine a baseline circadian pattern of blood pressure for a patient over a first period of time;
      determine a subsequent circadian pattern of blood pressure for the patient over a second period of time, the second period of time being after the first period of time;
      determine one or more differences between the subsequent circadian pattern and the baseline circadian pattern;
      determine, based on the one or more differences between the subsequent circadian pattern and the baseline circadian pattern, whether the patient is a candidate for denervation therapy; and
      output, responsive to determining whether the patient is a candidate for the denervation therapy, an indication of whether the patient is or is not a candidate for the denervation therapy.
2. The computing device of clause 1, wherein the one or more processors are further configured to:
   obtain one or more of the first blood pressure values or physiological parameters indicative of the first blood pressure values of the patient over the first period of time from a sensing device; and
   obtain one or more of the second blood pressure values or physiological parameters indicative of the second blood pressure values of the patient over the second period of time from the sensing device.
3. The computing device of any of clauses 1 through 2, wherein the one or more processors are further configured to:
   determine whether the differences between the subsequent circadian pattern and the baseline circadian pattern are greater than a circadian pattern difference threshold;
   responsive to determining the differences between the subsequent circadian pattern and baseline circadian pattern are greater than the circadian pattern difference threshold, output an indication that the patient is a candidate for a denervation therapy; and
   responsive to determining the differences between the subsequent circadian pattern and baseline circadian pattern are less than or equal to the circadian pattern difference threshold, output an indication that the patient is not a candidate for denervation therapy.
4. The computing device of any of clauses 1 through 3, wherein the one or more processors are further configured to:
   responsive to determining the differences between the subsequent circadian pattern and baseline circadian pattern are greater than the circadian pattern difference threshold, determine one or more of basal sympathetic nervous system activity is greater than a basal sympathetic nervous system activity threshold or change in basal sympathetic nervous system activity is greater than a change in basal sympathetic nervous system activity threshold;
   responsive to determining the differences between the subsequent circadian pattern and baseline circadian pattern are less than or equal to the circadian pattern difference threshold, determine one or more of the basal sympathetic nervous system activity is less than or equal to the basal sympathetic nervous system activity threshold or the change in basal sympathetic nervous system activity is less than or equal to the change in basal sympathetic nervous system activity threshold;
   responsive to determining one or more of the basal sympathetic nervous system activity is greater than the basal sympathetic nervous system activity threshold or the change in basal sympathetic nervous system activity is greater than the change in basal sympathetic nervous system activity threshold, output the indication that the patient is a candidate for a denervation therapy; and
   responsive to determining one or more of the basal sympathetic nervous system activity is less than or equal to the basal sympathetic nervous system activity threshold or the change in basal sympathetic nervous system activity is less than or equal to the change in basal sympathetic nervous system activity threshold, output the indication that the patient is a not candidate for denervation therapy.
5. The computing device of any of clauses 1 through 4, wherein the first period of time is one week, two weeks, or one month.
6. The computing device of any of clauses 1 through 5, wherein the second period of time is three days, five days, one week, or two weeks.
7. The computing device of any of clauses 1 through 6, wherein features of the baseline circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability, and
   features of the subsequent circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability.
8. The computing device of clause 7, wherein the one or more processors are further configured to compare one or more of the features of the baseline circadian pattern to a respective one or more of the features of the subsequent circadian pattern to determine the one or more differences between the subsequent circadian pattern and the baseline circadian pattern.
9. The computing device of any of clauses 1 through 8, wherein the first blood pressure values or physiological parameters indicative of the first blood pressure values and the second blood pressure values or physiological parameters indicative of the second blood pressure values are obtained from a wearable device.
10. The computing device of any of clauses 1 through 9, wherein the first blood pressure values and the second blood pressure values are systolic blood pressure values.
11. A system comprising:
   a wearable device configured to sense and collect data indicative of blood pressure of a patient; and
   the computing device of any one of claims 1-10, wherein the computing device is communicatively coupled to the wearable device.
12. A system comprising:
   a wearable device configured to sense and collect data indicative of blood pressure of a patient; and
   a computing device communicatively coupled to the wearable device, wherein the computing device comprises:
   a memory; and
   one or more processors coupled to the memory, the one or more processors being configured to:
   determine a baseline circadian pattern of blood pressure for a patient over a first period of time;
   determine a subsequent circadian pattern of blood pressure for the patient over a second period of time, the second period of time being after the first period of time;
   determine one or more differences between the subsequent circadian pattern and the baseline circadian pattern;
   determine, based on the one or more differences between the subsequent circadian pattern and the baseline circadian pattern, whether the patient is a candidate for denervation therapy; and
   output, responsive to determining whether the patient is a candidate for the denervation therapy, an indication of whether the patient is or is not a candidate for the denervation therapy.
13. The system of clause 12, wherein the one or more processors are further configured to:
   obtain one or more of the first blood pressure values or physiological parameters indicative of the first blood pressure values of the patient over the first period of time from a sensing device; and
   obtain one or more of the second blood pressure values or physiological parameters indicative of the second blood pressure values of the patient over the second period of time from the sensing device.
14. The system of any of clauses 12 through 13, wherein the one or more processors are further configured to:
   determine whether the differences between the subsequent circadian pattern and the baseline circadian pattern are greater than a circadian pattern difference threshold;
   responsive to determining the differences between the subsequent circadian pattern and baseline circadian pattern are greater than the circadian pattern difference threshold, output an indication that the patient is a candidate for a denervation therapy; and
   responsive to determining the differences between the subsequent circadian pattern and baseline circadian pattern are less than or equal to the circadian pattern difference threshold, output an indication that the patient is not a candidate for denervation therapy.
15. The system of any of clauses 12 through 14, wherein the one or more processors are further configured to:
   responsive to determining the differences between the subsequent circadian pattern and baseline circadian pattern are greater than the circadian pattern difference threshold, determine one or more of basal sympathetic nervous system activity is greater than a basal sympathetic nervous system activity threshold or change in basal sympathetic nervous system activity is greater than a change in basal sympathetic nervous system activity threshold;
   responsive to determining the differences between the subsequent circadian pattern and baseline circadian pattern are less than or equal to the circadian pattern difference threshold, determine one or more of the basal sympathetic nervous system activity is less than or equal to the basal sympathetic nervous system activity threshold or the change in basal sympathetic nervous system activity is less than or equal to the change in basal sympathetic nervous system activity threshold;
   responsive to determining one or more of the basal sympathetic nervous system activity is greater than the basal sympathetic nervous system activity threshold or the change in basal sympathetic nervous system activity is greater than the change in basal sympathetic nervous system activity threshold, output the indication that the patient is a candidate for a denervation therapy; and
   responsive to determining one or more of the basal sympathetic nervous system activity is less than or equal to the basal sympathetic nervous system activity threshold or the change in basal sympathetic nervous system activity is less than or equal to the change in basal sympathetic nervous system activity threshold, output the indication that the patient is a not candidate for denervation therapy.
16. The system of any of clauses 12 through 14, wherein the first period of time is one week, two weeks, or one month.
17. The system of any of clauses 12 through 16, wherein the second period of time is three days, five days, one week, or two weeks.
18. The system of any of clauses 12 through 17, wherein features of the baseline circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability, and
   features of the subsequent circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability.
19. The system of clause 18, wherein the one or more processors are further configured to compare one or more of the features of the baseline circadian pattern to a respective one or more of the features of the subsequent circadian pattern to determine the one or more differences between the subsequent circadian pattern and the baseline circadian pattern.
20. The system of any of clauses 12 through 19, wherein the first blood pressure values or physiological parameters indicative of the first blood pressure values and the second blood pressure values or physiological parameters indicative of the second blood pressure values are obtained from a wearable device.
21. The system of any of clauses 12 through 20, wherein the first blood pressure values and the second blood pressure values are systolic blood pressure values 22. A method comprising:
   determining a baseline circadian pattern of blood pressure for a patient over a first period of time;
   determining a subsequent circadian pattern of blood pressure for the patient over a second period of time, the second period of time being after the first period of time;
   determining one or more differences between the subsequent circadian pattern and the baseline circadian pattern;
   determining, based on the one or more differences between the subsequent circadian pattern and the baseline circadian pattern, whether the patient is a candidate for denervation therapy; and
   outputting, responsive to determining whether the patient is a candidate for the denervation therapy, an indication of whether the patient is or is not a candidate for the denervation therapy.
23. The method of clause 22, wherein the method further comprises:
   obtaining one or more of the first blood pressure values or physiological parameters indicative of the first blood pressure values of the patient over the first period of time from a sensing device; and
   obtaining one or more of the second blood pressure values or physiological parameters indicative of the second blood pressure values of the patient over the second period of time from the sensing device.
24. The method of any one of clauses 22 or 23, wherein the method further comprises:
   determining whether the differences between the subsequent circadian pattern and the baseline circadian pattern are greater than a circadian pattern difference threshold;
   responsive to determining the differences between the subsequent circadian pattern and baseline circadian pattern are greater than the circadian pattern difference threshold, outputting an indication that the patient is a candidate for a denervation therapy; and
   responsive to determining the differences between the subsequent circadian pattern and baseline circadian pattern are less than or equal to the circadian pattern difference threshold, outputting an indication that the patient is not a candidate for denervation therapy.
25. The method of clauses 22 through 24, wherein the method further comprises:
   responsive to determining the differences between the subsequent circadian pattern and baseline circadian pattern are greater than the circadian pattern difference threshold, determining one or more of basal sympathetic nervous system activity is greater than a basal sympathetic nervous system activity threshold or change in basal sympathetic nervous system activity is greater than a change in basal sympathetic nervous system activity threshold;
   responsive to determining the differences between the subsequent circadian pattern and baseline circadian pattern are less than or equal to the circadian pattern difference threshold, determining one or more of the basal sympathetic nervous system activity is less than or equal to the basal sympathetic nervous system activity threshold or the change in basal sympathetic nervous system activity is less than or equal to the change in basal sympathetic nervous system activity threshold;
   responsive to determining one or more of the basal sympathetic nervous system activity is greater than the basal sympathetic nervous system activity threshold or the change in basal sympathetic nervous system activity is greater than the change in basal sympathetic nervous system activity threshold, outputting the indication that the patient is a candidate for a denervation therapy; and
   responsive to determining one or more of the basal sympathetic nervous system activity is less than or equal to the basal sympathetic nervous system activity threshold or the change in basal sympathetic nervous system activity is less than or equal to the change in basal sympathetic nervous system activity threshold, outputting the indication that the patient is a not candidate for denervation therapy.
26. The method of any of clauses 22 through 25, wherein the first period of time is one week, two weeks, or one month.
27. The method of any of clauses 22 through 26, wherein the second period of time is three days, five days, one week, or two weeks.
28. The method of any of clauses 22 through 27, wherein features of the baseline circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability, and
   features of the subsequent circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability.
29. The method of clause 28, wherein the method further comprises comparing one or more of the features of the baseline circadian pattern to a respective one or more of the features of the subsequent circadian pattern to determine the one or more differences between the subsequent circadian pattern and the baseline circadian pattern.
30. The method of any of clauses 22 through 29, wherein the first blood pressure values or physiological parameters indicative of the first blood pressure values and the second blood pressure values or physiological parameters indicative of the second blood pressure values are obtained from a wearable device.
31. The method of any of clauses 22 through 30, wherein the first blood pressure values and the second blood pressure values are systolic blood pressure values.

1. A computing device comprising:
a memory; and
one or more processors coupled to the memory, the one or more processors being configured to:
determine a baseline circadian pattern of blood pressure for a patient over a first period of time;
determine a subsequent circadian pattern of blood pressure for the patient over a second period of time, the second period of time being after the first period of time;
determine one or more differences between the subsequent circadian pattern and the baseline circadian pattern;
determine, based on the one or more differences between the subsequent circadian pattern and the baseline circadian pattern, whether the patient is a candidate for denervation therapy; and
output, responsive to determining whether the patient is a candidate for the denervation therapy, an indication of whether the patient is or is not a candidate for the denervation therapy.

2. The computing device of claim 1, wherein the one or more processors are further configured to:
obtain one or more of the first blood pressure values or physiological parameters indicative of the first blood pressure values of the patient over the first period of time from a sensing device; and
obtain one or more of the second blood pressure values or physiological parameters indicative of the second blood pressure values of the patient over the second period of time from the sensing device.

3. The computing device of claim 1, wherein the one or more processors are further configured to:
determine whether the differences between the subsequent circadian pattern and the baseline circadian pattern are greater than a circadian pattern difference threshold;
responsive to determining the differences between the subsequent circadian pattern and baseline circadian pattern are greater than the circadian pattern difference threshold, output an indication that the patient is a candidate for a denervation therapy; and
responsive to determining the differences between the subsequent circadian pattern and baseline circadian pattern are less than or equal to the circadian pattern difference threshold, output an indication that the patient is not a candidate for denervation therapy.

4. The computing device of claim 1, wherein the one or more processors are further configured to:
responsive to determining the differences between the subsequent circadian pattern and baseline circadian pattern are greater than the circadian pattern difference threshold, determine one or more of basal sympathetic nervous system activity is greater than a basal sympathetic nervous system activity threshold or change in basal sympathetic nervous system activity is greater than a change in basal sympathetic nervous system activity threshold;
responsive to determining the differences between the subsequent circadian pattern and baseline circadian pattern are less than or equal to the circadian pattern difference threshold, determine one or more of the basal sympathetic nervous system activity is less than or equal to the basal sympathetic nervous system activity threshold or the change in basal sympathetic nervous system activity is less than or equal to the change in basal sympathetic nervous system activity threshold;
responsive to determining one or more of the basal sympathetic nervous system activity is greater than the basal sympathetic nervous system activity threshold or the change in basal sympathetic nervous system activity is greater than the change in basal sympathetic nervous system activity threshold, output the indication that the patient is a candidate for a denervation therapy; and
responsive to determining one or more of the basal sympathetic nervous system activity is less than or equal to the basal sympathetic nervous system activity threshold or the change in basal sympathetic nervous system activity is less than or equal to the change in basal sympathetic nervous system activity threshold, output the indication that the patient is a not candidate for denervation therapy.

5. The computing device of claim 1, wherein the first period of time is one week, two weeks, or one month.

6. The computing device of claim 1, wherein the second period of time is three days, five days, one week, or two weeks.

7. The computing device of claim 1, wherein features of the baseline circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability, and
features of the subsequent circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability.

8. The computing device of claim 7, wherein the one or more processors are further configured to compare one or more of the features of the baseline circadian pattern to a respective one or more of the features of the subsequent circadian pattern to determine the one or more differences between the subsequent circadian pattern and the baseline circadian pattern.

9. The computing device of claim 1, wherein the first blood pressure values or physiological parameters indicative of the first blood pressure values and the second blood pressure values or physiological parameters indicative of the second blood pressure values are obtained from a wearable device.

10. The computing device of claim 1, wherein the first blood pressure values and the second blood pressure values are systolic blood pressure values.

11. A system comprising:
a wearable device configured to sense and collect data indicative of blood pressure of a patient; and
a computing device comprising:
a memory; and
one or more processors coupled to the memory, the one or more processors being configured to:
determine a baseline circadian pattern of blood pressure for the patient over a first period of time;
determine a subsequent circadian pattern of blood pressure for the patient over a second period of time, the second period of time being after the first period of time;
determine one or more differences between the subsequent circadian pattern and the baseline circadian pattern;
determine, based on the one or more differences between the subsequent circadian pattern and the baseline circadian pattern, whether the patient is a candidate for denervation therapy; and
output, responsive to determining whether the patient is a candidate for the denervation therapy, an indication of whether the patient is or is not a candidate for the denervation therapy,
wherein the computing device is communicatively coupled to the wearable device.

12. A method comprising:
determining a baseline circadian pattern of blood pressure for a patient over a first period of time;
determining a subsequent circadian pattern of blood pressure for the patient over a second period of time, the second period of time being after the first period of time;
determining one or more differences between the subsequent circadian pattern and the baseline circadian pattern;
determining, based on the one or more differences between the subsequent circadian pattern and the baseline circadian pattern, whether the patient is a candidate for denervation therapy; and
outputting, responsive to determining whether the patient is a candidate for the denervation therapy, an indication of whether the patient is or is not a candidate for the denervation therapy.

13. The method of claim 12, wherein the method further comprises:
obtaining one or more of the first blood pressure values or physiological parameters indicative of the first blood pressure values of the patient over the first period of time from a sensing device; and
obtaining one or more of the second blood pressure values or physiological parameters indicative of the second blood pressure values of the patient over the second period of time from the sensing device.

14. The method of claim 12, wherein the method further comprises:
determining whether the differences between the subsequent circadian pattern and the baseline circadian pattern are greater than a circadian pattern difference threshold;
responsive to determining the differences between the subsequent circadian pattern and baseline circadian pattern are greater than the circadian pattern difference threshold, outputting an indication that the patient is a candidate for a denervation therapy; and
responsive to determining the differences between the subsequent circadian pattern and baseline circadian pattern are less than or equal to the circadian pattern difference threshold, outputting an indication that the patient is not a candidate for denervation therapy.

15. The method of claim 14, wherein the method further comprises:
responsive to determining the differences between the subsequent circadian pattern and baseline circadian pattern are greater than the circadian pattern difference threshold, determining one or more of basal sympathetic nervous system activity is above basal sympathetic nervous system activity threshold or change in basal sympathetic nervous system activity is above a change in basal sympathetic nervous system activity threshold; and
responsive to determining the differences between the subsequent circadian pattern and baseline circadian pattern are less than or equal to the circadian pattern difference threshold, determining one or more of the basal sympathetic nervous system activity is below the basal sympathetic nervous system activity threshold or the change in basal sympathetic nervous system activity is below the change in basal sympathetic nervous system activity threshold.

16. The method of claim 15, wherein the method further comprises:
responsive to determining one or more of the basal sympathetic nervous system activity is above the basal sympathetic nervous system activity threshold or the change in basal sympathetic nervous system activity is above the change in basal sympathetic nervous system activity threshold, outputting the indication that the patient is a candidate for a denervation therapy; and
responsive to the computing device determining one or more of the basal sympathetic nervous system activity is below the basal sympathetic nervous system activity threshold or the change in basal sympathetic nervous system activity is below the change in basal sympathetic nervous system activity threshold, outputting the indication that the patient is a not candidate for denervation therapy.

17. The method of claim 12, wherein the first period of time is up to one week, two weeks, or one month, and the second period of time is up to three days, five days, one week, or two weeks.

18. The method of claim 12, wherein the second period of time is a moving average.

19. The method of claim 12, wherein features of the baseline circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability; and
features of the subsequent circadian pattern include at least one of a slope of a morning surge, a slope of an evening surge, an amplitude of a nighttime blood pressure drop, an amplitude of an evening blood pressure surge, a daytime variability, a nighttime variability, or a 24-hour variability.

20. The method of claim 19, further comprising:
comparing one or more of the features of the baseline circadian pattern to a respective one or more of the features of the subsequent circadian pattern to determine the one or more differences between the subsequent circadian pattern and the baseline circadian pattern.
